SEQUENCE LISTING (5 sequences)

SEQ ID NO: 1 - DNA, 22 bp, synthetic construct
  misc_feature 1..22: note = Synthetic Construct
  source 1..22: mol_type = other DNA
tgactgtgaacgttcgagatga

SEQ ID NO: 2 - DNA, 11 bp, synthetic construct
  misc_feature 1..11: note = Synthetic Construct
  modified_base 3: mod_base = OTHER; note = 2'-deoxy-7-deazaguanosine
  modified_base 7: mod_base = OTHER; note = 2'-deoxy-7-deazaguanosine
  modified_base 11: mod_base = OTHER; note = 2'-deoxy-7-deazaguanosine
  source 1..11: mol_type = other DNA
tcgaacgttcg

SEQ ID NO: 3 - DNA, 10 bp, synthetic construct
  misc_feature 1..10: note = Synthetic Construct
  source 1..10: mol_type = other DNA
aacgttcgag

SEQ ID NO: 4 - protein, 546 aa, Varicella zoster virus
MGTVNKPVVGVLMGFGIITGTLRITNPVRASVLRYDDFHIDEDKLDTNSVYEPYYHSDHAESSWVNRGESSRKAYDHNSPYIWPRNDYDGFLENAHEHHGVYNQGRGIDSGERLMQPTQMSAQEDLGDDTGIHVIPTLNGDDRHKIVNVDQRQYGDVFKGDLNPKPQGQRLIEVSVEENHPFTLRAPIQRIYGVRYTETWSFLPSLTCTGDAAPAIQHICLKHTTCFQDVVVDVDCAENTKEDQLAEISYRFQGKKEADQPWIVVNTSTLFDELELDPPEIEPGVLKVLRTEKQYLGVYIWNMRGSDGTSTYATFLVTWKGDEKTRNPTPAVTPQPRGAEFHMWNYHSHVFSVGDTFSLAMHLQYKIHEAPFDLLLEWLYVPIDPTCQPMRLYSTCLYHPNAPQCLSHMNSGCTFTSPHLAQRVASTVYQNCEHADNYTAYCLGISHMEPSFGLILHDGGTTLKFVDTPESLSGLYVFVVYFNGHVEAVAYTVVSTVDHFVNAIEERGFPPTAGQPPATTKPKEITPVNPGTSPLIRYAAWTGGLA

SEQ ID NO: 5 - protein, 537 aa, Varicella zoster virus
MGTVNKPVVGVLMGFGIITGTLRITNPVRASVLRYDDFHTDEDKLDTNSVYEPYYHSDHAESSWVNRGESSRKAYDHNSPYIWPRNDYDGFLENAHEHHGVYNQGRGIDSGERLMQPTQMSAQEDLGDDTGIHVIPTLNGDDRHKIVNVDQRQYGDVFKGDLNPKPQGQRLIEVSVEENHPFTLRAPIQRIYGVRYTETWSFLPSLTCTGDAAPAIQHICLKHTTCFQDVVVDVDCAENTKEDQLAEISYRFQGKKEADQPWIVVNTSTLFDELELDPPEIEPGVLKVLRTEKQYLGVYIWNMRGSDGTSTYATFLVTWKGDEKTRNPTPAVTPQPRGAEFHMWNYHSHVFSVGDTFSLAMHLQYKIHEAPFDLLLEWLYVPIDPTCQPMRLYSTCLYHPNAPQCLSHMNSGCTFTSPHLAQRVASTVYQNCEHADNYTAYCLGISHMEPSFGLILHDGGTTLKFVDTPESLSGLYVFVVYFNGHVEAVAYTVVSTVDHFVNAIEERGFPPTAGQPPATTKPKEITPVNPGTSPLLR

(19) United States
(12) Patent Application Publication
(10) Pub. No.: US 2026/0315587 A1
(43) Pub. Date: Oct. 15, 2026

(54) SHINGLES VACCINE COMPRISING A TLR9 AGONIST

(71) Applicant: Dynavax Technologies Corporation, Emeryville, CA (US)

(72) Inventors: John D. CAMPBELL, San Francisco, CA (US); Randall N. HYER, Gwynedd Valley, PA (US); Robert S. JANSSEN, Oakland, CA (US); David NOVACK, Oakland, CA (US)

(73) Assignee: Dynavax Technologies Corporation, Emeryville, CA (US)

(21) Appl. No.: 19/533,015

(22) Filed: Feb. 6, 2026

Related U.S. Application Data
(63) Continuation of application No. 17/910,334, filed on Mar. 9, 2021, now Pat. No. 12,569,553.
(60) Provisional application No. 62/987,243, filed on Mar. 9, 2020.

(51) Int. Cl. A61K 39/25; A61K 39/00; A61P 31/22

(52) U.S. Cl. CPC A61K 39/25; A61P 31/22; A61K 2039/545; A61K 2039/55505; A61K 2039/55561

(57) ABSTRACT

The present disclosure relates to immunogenic compositions comprising a varicella zoster virus (VZV) glycoprotein E antigen and a toll-like receptor 9 (TLR9) agonist, such as an oligonucleotide comprising an unmethylated cytidine-phospho-guanosine (CpG) motif. The immunogenic compositions are suitable for stimulating an immune response against VZV in an individual in need thereof.

### CROSS-REFERENCE TO RELATED APPLICATIONS

This application is a continuation of U.S. patent application Ser. No. 17/910,334, filed Sep. 8, 2022, which is a national stage application under 35 U.S.C. § 371 of International Application No. PCT/US2021/021544, filed Mar. 9, 2021, which claims priority to and the benefit of U.S. Provisional Application No. 62/987,243, filed Mar. 9, 2020, the disclosure of each of which is hereby incorporated by reference in its entirety.

### SUBMISSION OF SEQUENCE LISTING AS ASCII TEXT FILE

The content of the following submission on ASCII text file is incorporated herein by reference in its entirety: a computer readable form (CRF) of the Sequence Listing (377882007101seglist.xml; Size: 9,264 bytes; and Date of Creation: Feb. 6, 2026).

### FIELD

The present disclosure relates to immunogenic compositions comprising a varicella zoster virus surface (VZV) glycoprotein E antigen and a toll-like receptor 9 (TLR9) agonist, such as an oligonucleotide comprising an unmethylated cytidine-phospho-guanosine (CpG) motif. The immunogenic compositions are suitable for stimulating an immune response against VZV in an individual in need thereof.

### BACKGROUND

Shingles (herpes zoster) is a viral disease characterized by a feeling of burning, tingling and itching on one side of the body or face, followed by a painful rash and blisters. According to the Center for Disease Control and Prevention (CDC), about 10-13% of older people with shingles experience long-term nerve pain (postherpetic neuralgia) as a complication. Additionally, a shingles rash near an eye or an ear may result in vision loss or cause muscles of the face to become paralyzed (Ramsay Hunt syndrome).

Shingles, like chicken pox, is caused by infection with varicella zoster virus (VZV). Shingles is caused by reactivation of latent VZV in an individual who had chickenpox as a result of an initial infection with VZV. Prior to U.S. Food and Drug Administration (FDA) approval of a varicella vaccine in 1995, most children in the U.S. contracted chickenpox and are therefore now at risk of contracting shingles as adults.

Two vaccines are available for prevention of herpes zoster in individuals who are 50 years of age or older. ZOSTAVAX® is a live attenuated virus vaccine marketed by Merck & Co., Inc. (Whitehouse Station, NJ). SHINGRIX® is a recombinant, adjuvanted subunit vaccine marketed by GlaxoSmithKline (Research Triangle Park, NC). Although no head-to-head clinical trials were conducted, the Advisory Committee on Immunization Practices narrowly voted in 2017 to recommend preferential use of SHINGRIX® over ZOSTAVAX® based on estimates of improved efficacy against herpes zoster and postherpetic neuralgia (Dooling et al., MMWR, 67:103-108, 2018). SHINGRIX® contains a recombinant VZV glycoprotein E (gE) as an antigen in combination with ASO1B as the adjuvant. ASO1B is composed of 3-O-desacyl-4′-monophosphoryl lipid A (MPL) from Salmonella minnesota and QS-21, a saponin purified from an extract of Quillaja saponaria Molina, combined in a liposomal formulation. The narrow vote in favor of SHINGRIX® (8 to 7) was associated with concerns about reactogenicity. Phase III studies for SHINGRIX® reported that 16.5% of vaccine recipients developed grade 3 adverse events versus 3.1% of placebo recipients, and that 10.8% of vaccine recipients developed grade 3 systemic events (myalgia, fatigue, headache, shivering, fever, and gastrointestinal symptoms) versus 2.4% of placebo recipients (Lal et al., N Eng J Med, 372:2087-2096, 2015; and Cunningham et al., N Eng J Med, 375:1019-1032, 2016). The relatively high levels of serious side effects after SHINGRIX® vaccination was recognized as having the potential to negatively impact second dose compliance levels in patients (Bharucha et al., Human Vaccines & Immunotherapeutics, 13:1789-1797, 2017), and this has been a persistent concern given the need for a patient to receive both doses to be protected against shingles.

Reactogenicity of protein subunit vaccines such as SHINGRIX® can be influenced by a variety of factors, chief among which is the nature of the adjuvant included to improve immunogenicity. Different Adjuvant Systems (AS) adjuvants combined with a model protein antigen were compared by immunization of healthy, human subjects. The AS01B adjuvant was found to induce higher levels of local and systemic reactogenicity than the comparator adjuvants (AS01E, AS03A, AS04, and alum)(Leroux-Roels et al., Clin Immunol, 169:16-27, 2016). This suggests that AS01B is contributing significantly to the concerning reactogenicity profile of SHINGRIX.

Thus, there is a need for an improved Shingles vaccine with a superior safety profile, but which is not inferior to SHINGRIX® with regard to VZV gE immunogenicity.

### SUMMARY

The present disclosure relates to immunogenic compositions comprising a varicella zoster virus surface (VZV) glycoprotein E antigen and a toll-like receptor 9 (TLR9) agonist, such as an oligonucleotide comprising an unmethylated cytidine-phospho-guanosine (CpG) motif. The immunogenic compositions are suitable for stimulating an immune response against VZV in individuals in need thereof.

### BRIEF DESCRIPTION OF THE DRAWINGS

FIG. 1A shows the levels of anti-VZV gE-reactive IgG in sera of recipients of a first dose (prime) of a shingles vaccine and control recipients (CTL and LAV). FIG. 1B shows the levels of anti-VZV gE-reactive IgG in sera of recipients of a first dose (prime) and a second dose (boost) of a shingles vaccine and control recipients (CTL and LAV).

FIG. 2A-2C show gE-antigen induced, IL-2 and/or IFN-γ secreting CD4+ T cells of recipients of a first dose (prime) and a second dose (boost) of a shingles vaccine and control recipients (CTL and LAV).

FIG. 3A-3B show gE-antigen induced, IL-2 or IFN-γ secreting CD154+, CD4+ T cells of recipients of a first dose (prime) and a second dose (boost) of a shingles vaccine and control recipients (CTL and LAV).

### GENERAL TECHNIQUES AND DEFINITIONS

The practice of the present disclosure will employ, unless otherwise indicated, conventional techniques of molecular biology (including recombinant techniques), microbiology, cell biology, biochemistry and immunology, which are within the skill of the art.

As used herein and in the appended claims, the singular forms “a”, “an”, and “the” include plural references unless indicated otherwise. For example, “an” excipient includes one or more excipients.

The phrase “comprising” as used herein is open-ended, indicating that such embodiments may include additional elements. In contrast, the phrase “consisting of” is closed, indicating that such embodiments do not include additional elements (except for trace impurities). The phrase “consisting essentially of” is partially closed, indicating that such embodiments may further comprise elements that do not materially change the basic characteristics of such embodiments.

The term “about” as used herein in reference to a value, encompasses from 90% to 110% of that value (e.g., about 3000 μg of CpG 1018 refers to 2700 μg to 3300 μg of CpG 1018).

As used interchangeably herein, the terms “polynucleotide” and “oligonucleotide” include single-stranded DNA (ssDNA), double-stranded DNA (dsDNA), single-stranded RNA (ssRNA) and double-stranded RNA (dsRNA), modified oligonucleotides and oligonucleosides or combinations thereof. The oligonucleotide can be linearly or circularly configured, or the oligonucleotide can contain both linear and circular segments. Oligonucleotides are polymers of nucleosides joined, generally, through phosphodiester linkages, although alternate linkages, such as phosphorothioate esters may also be used in oligonucleotides. A nucleoside consists of a purine (adenine (A) or guanine (G) or derivative thereof) or pyrimidine (thymine (T), cytosine (C) or uracil (U), or derivative thereof) base bonded to a sugar. The four nucleoside units (or bases) in DNA are called deoxyadenosine, deoxyguanosine, thymidine, and deoxycytidine. A nucleotide is a phosphate ester of a nucleoside.

The terms “CpG,” “CpG motif,” and “cytosine-phosphate-guanosine,” as used herein, refer to an unmethylated cytidine-phospho-guanosine dinucleotide, which when present in an oligonucleotide contributes to a measurable immune response in vitro, in vivo and/or ex vivo. Examples of measurable immune responses include, but are not limited to, antigen-specific antibody production, secretion of cytokines, activation or expansion of lymphocyte populations, such as NK cells, CD4+ T lymphocytes, CD8+ T lymphocytes, B lymphocytes, and the like. Preferably, the CpG oligonucleotide preferentially activates a Th1-type response.

An “effective amount” or a “sufficient amount” of a substance is that amount sufficient to effect beneficial or desired results, including clinical results, and, as such, an “effective amount” depends upon the context in which it is being applied. In the context of administering an immunogenic composition, an effective amount contains sufficient antigen and TLR9 agonist to stimulate an immune response (preferably a seroprotective level of antibody to the antigen).

The terms “individual” and “subject” refer to mammals. “Mammals” include, but are not limited to, humans, non-human primates (e.g., monkeys), farm animals, sport animals, rodents (e.g., mice and rats), and pets (e.g., dogs and cats).

The term “dose” as used herein in reference to an immunogenic composition refers to a measured portion of the immunogenic composition taken by (administered to or received by) a subject at any one time.

The terms “isolated” and “purified” as used herein refers to a material that is removed from at least one component with which it is naturally associated (e.g., removed from its original environment). The term “isolated,” when used in reference to a recombinant protein, refers to a protein that has been removed from the culture medium of the host cell that produced the protein.

“Stimulation” of a response or parameter includes eliciting and/or enhancing that response or parameter when compared to otherwise same conditions except for a parameter of interest, or alternatively, as compared to another condition (e.g., increase in TLR-signaling in the presence of a TLR agonist as compared to the absence of the TLR agonist). For example, “stimulation” of an immune response means an increase in the response. Depending upon the parameter measured, the increase may be from 5-fold to 500-fold or over, or from 5, 10, 50, or 100-fold to 500, 1,000, 5,000, or 10,000-fold.

As used herein the term “immunization” refers to a process that increases a mammalian subject's reaction to antigen and therefore improves its ability to resist or overcome infection and/or resist disease.

The term “vaccination” as used herein refers to the introduction of vaccine into a body of a mammalian subject.

“Adjuvant” refers to a substance which, when added to a composition comprising an antigen, enhances or potentiates an immune response to the antigen in the mammalian recipient upon exposure.

### DETAILED DESCRIPTION

The present disclosure relates to immunogenic compositions comprising a varicella zoster virus (VZV) glycoprotein E (gE) antigen and a toll-like receptor 9 (TLR9) agonist, such as oligonucleotide comprising an unmethylated cytidine-phospho-guanosine (CpG) motif. The VZV gE antigen may be a truncated recombinant protein devoid of signal peptide, transmembrane and cytoplasmic domains of a full-length VZV gE antigen. The immunogenic compositions are suitable for stimulating an immune response against varicella zoster virus in an individual in need thereof.

### I. Immunogenic Compositions and Kits

The present disclosure relates to immunogenic compositions for stimulating an immune response against varicella zoster virus (VZV), comprising a VZV glycoprotein E (gE) antigen and a toll-like receptor 9 (TLR9) agonist, wherein the TLR9 agonist is an oligonucleotide of from 8 to 35 nucleotides in length comprising an unmethylated cytidine-phospho-guanosine (also referred to as CpG or cytosine-phosphate-guanosine) motif, and the gE antigen and oligonucleotide are present in the immunogenic composition in amounts effective to stimulate an immune response against the gE antigen in a mammalian subject, such as a human) subject. In some embodiments, the immunogenic compositions further comprise an aluminum salt adjuvant to which the VZV gE antigen is adsorbed.

### A. Toll-Like Receptor 9 (TLR9) Agonists

Toll-like receptors (TLRs) are expressed in and on dendritic cells and other innate immune cells and are among the most important receptors for stimulating a response to the presence of invading pathogens. Humans have multiple types of TLRs that are similar in structure but recognize different parts of viruses or bacteria. By activating specific TLRs, it is possible to stimulate and control specific types of innate immune responses that can be harnessed to enhance adaptive responses.

TLR9 (CD289) recognizes unmethylated cytidine-phospho-guanosine (CpG) motifs found in microbial DNA, which can be mimicked using synthetic CpG-containing oligodeoxynucleotides (CpG-ODNs). CpG-ODNs are known to enhance antibody production and to stimulate T helper 1 (Th1) cell responses (Coffman et al., Immunity, 33:492-503, 2010). Based on structure and biological function, CpG-ODNs have been divided into three general classes: CpG-A, CpG-B, and CpG-C(Campbell, Methods Mol Biol, 1494:15-27, 2017). The degree of B cell activation varies between the classes with CpG-A ODNs being weak, CpG-C ODNs being good, and CpG-B ODNs being strong B cell activators. Oligonucleotide TLR9 agonists of the present disclosure are preferably good B cell activators (CpG-C ODN) or more preferably strong (CpG-B ODN) B cell activators.

Optimal oligonucleotide TLR9 agonists often contain a palindromic sequence following the general formula of: 5′-purine-purine-CG-pyrimidine-pyrimidine-3′, or 5′-purine-purine-CG-pyrimidine-pyrimidine-CG-3′ (U.S. Pat. No. 6,589,940). TLR9 agonism is also observed with certain non-palindromic CpG-enriched phosphorothioate oligonucleotides, but may be affected by changes in the nucleotide sequence. Additionally, TLR9 agonism is abolished by methylation of the cytosine within the CpG dinucleotide. Accordingly in some embodiments, the TLR9 agonist is an oligonucleotide of from 8 to 35 nucleotides in length comprising the sequence 5′-AACGTTCG-3′. In some embodiments, the oligonucleotide is greater than 8, 9, 10, 11, 12, 13, 14, 15, 16, 17, 18, 19, or 20 nucleotides in length, and the oligonucleotide is less than 35, 34, 33, 32, 31, 30, 29, 28, 27, 26, 25, or 24 nucleotides in length. In some embodiments, the TLR9 agonist is an oligonucleotide of from 10 to 35 nucleotides in length comprising the sequence 5′-AACGTTCGAG-3′ (SEQ ID NO:3). In some embodiments, the oligonucleotide is greater than 10, 11, 12, 13, 14, 15, 16, 17, 18, 19, or 20 nucleotides in length, and the oligonucleotide is less than 35, 34, 33, 32, 31, 30, 29, 28, 27, 26, 25, or 24 nucleotides in length.

Researchers at Dynavax Technologies Corporation (Emeryville, CA) have identified a 22-mer phosphorothioate linked oligodeoxynucleotide, CpG 1018, which contains specific sequences that can substantially enhance the immune response to co-administered antigens across species (Campbell, Methods Mol Biol, 1494:15-27, 2017). CpG 1018 (5′-TGACTGTGAA CGTTCGAGAT GA-3′, set forth as SEQ ID NO:1) was chosen after screening a broad panel of oligonucleotides for immunostimulatory activity in vitro and in vivo. CpG 1018 is a CpG-B ODN that is active in mice, rabbits, dogs, baboons, cynomolgus monkeys, and humans. Thus in some preferred embodiments, the TLR9 agonist is an oligonucleotide comprising the sequence of SEQ ID NO:1.

Although the exemplary oligonucleotide TLR9 agonist, CpG 1018, is a CpG-ODN, the present disclosure is not restricted to fully DNA molecules. That is, in some embodiments, the TLR9 agonist is a DNA/RNA chimeric molecule in which the CpG(s) and the palindromic sequence are deoxyribonucleic acids and one or more nucleic acids outside of these regions are ribonucleic acids. In some embodiments, the CpG oligonucleotide is linear. In other embodiments, the CpG oligonucleotide is circular or includes hairpin loop(s). The CpG oligonucleotide may be single stranded or double stranded.

In some embodiments, the CpG oligonucleotide may contain modifications. Modifications include but are not limited to, modifications of the 3′OH or 5′OH group, modifications of the nucleotide base, modifications of the sugar component, and modifications of the phosphate group. Modified bases may be included in the palindromic sequence of the CpG oligonucleotide as long as the modified base(s) maintains the same specificity for its natural complement through Watson-Crick base pairing (e.g., the palindromic portion is still self-complementary). In some embodiments, the CpG oligonucleotide comprises a non-canonical base. In some embodiments, the CpG oligonucleotide comprises a modified nucleoside. In some embodiments, the modified nucleoside is selected from the group consisting of 2′-deoxy-7-deazaguanosine, 2′-deoxy-6-thioguanosine, arabinoguanosine, 2′-deoxy-2′substituted-arabinoguanosine, and 2′-O-substituted-arabinoguanosine. In some embodiments, the TLR9 agonist is an oligonucleotide comprising the sequence 5′-TCG₁AACG₁TTCG₁-3′ (SEQ ID NO:2), in which G₁ is 2′-deoxy-7-deazaguanosine. In some embodiments, the oligonucleotide comprises the sequence 5′-TCG₁AACG₁TTCG₁-X-G₁CTTG₁CAAG₁CT-5′, and in which G₁ is 2′-deoxy-7-deazaguanosine and X is glycerol (5′-SEQ ID NO:2-3′-X-3′-SEQ ID NO:2-5′).

The CpG oligonucleotide may contain a modification of the phosphate group. For example, in addition to phosphodiester linkages, phosphate modifications include, but are not limited to, methyl phosphonate, phosphorothioate, phosphoramidate (bridging or non-bridging), phosphotriester and phosphorodithioate and may be used in any combination. Other non-phosphate linkages may also be used. In some embodiments, the oligonucleotides comprise only phosphorothioate backbones. In some embodiments, the oligonucleotides comprise only phosphodiester backbones. In some embodiments, the oligonucleotide comprises a combination of phosphate linkages in the phosphate backbone such as a combination of phosphodiester and phosphorothioate linkages. Oligonucleotides with phosphorothioate backbones can be more immunogenic than those with phosphodiester backbones and appear to be more resistant to degradation after injection into the host (Braun et al., J Immunol, 141:2084-2089, 1988; and Latimer et al., Mol Immunol, 32:1057-1064, 1995). The CpG oligonucleotides of the present disclosure include at least one, two or three internucleotide phosphorothioate ester linkages. In some embodiments, when a plurality of CpG oligonucleotide molecules are present in a pharmaceutical composition comprising at least one excipient, both stereoisomers of the phosphorothioate ester linkage are present in the plurality of CpG oligonucleotide molecules. In some embodiments, all of the internucleotide linkages of the CpG oligonucleotide are phosphorothioate linkages, or said another way, the CpG oligonucleotide has a phosphorothioate backbone.

A unit dose of the immunogenic composition, which is typically a 0.5 ml dose, may comprises from about 375 μg to about 6000 μg of the CpG oligonucleotide, preferably from about 750 μg to about 3000 μg of the CpG oligonucleotide. In some embodiments, a 0.5 ml dose of the immunogenic composition comprises greater than about 250, 500, 750, 1000, or 1250 μg of the CpG oligonucleotide, and less than about 6000, 5000, 4000, 3000, or 2000 μg of the CpG oligonucleotide. In some embodiments, a 0.5 ml dose of the immunogenic composition comprises about 375, 750, 1500, 3000 or 6000 μg of the CpG oligonucleotide. In some embodiments, a 0.5 ml dose of the immunogenic composition comprises about 750 μg of the CpG oligonucleotide. In some embodiments, a 0.5 ml dose of the immunogenic composition comprises about 1500 μg of the CpG oligonucleotide. In some embodiments, a 0.5 ml dose of the immunogenic composition comprises about 3000 μg of the CpG oligonucleotide.

The CpG oligonucleotides described herein are in their pharmaceutically acceptable salt form unless otherwise indicated. Exemplary basic salts include ammonium salts, alkali metal salts such as sodium, lithium, and potassium salts, alkaline earth metal salts such as calcium and magnesium salts, zinc salts, salts with organic bases (for example, organic amines) such as N-Me-D-glucamine, N-[1-(2,3-dioleoyloxy)propyl]-N,N,N-trimethylammonium chloride, choline, tromethamine, dicyclohexylamines, t-butyl amines, and salts with amino acids such as arginine, lysine and the like. In some embodiment, the CpG oligonucleotides are in the ammonium, sodium, lithium, or potassium salt form. In one preferred embodiment, the CpG oligonucleotides are in the sodium salt form.

### B. Varicella Zoster Virus (VZV) Glycoprotein E (gE) Antigen

A VZV gE antigen of the immunogenic compositions of the present disclosure comprises gE or a fragment thereof. In preferred embodiments, the gE antigen is recognized by VZV-reactive antibodies and/or peptide fragments of gE are recognized by VZV-reactive T cells. In some embodiments, the gE antigen is a recombinant protein, while in other embodiments the gE antigen is a purified from VZV virions. In some preferred embodiments, the gE antigen is an isolated antigen. In some embodiments, the VZV gE antigen is a truncated recombinant protein devoid of signal peptide, transmembrane and cytoplasmic domains of a full-length VZV gE antigen. The amino acid sequence of a representative gE is set forth as GenBank No. AQT34120.1. In some embodiments, the gE antigen comprises the amino acid sequence from residues 39-585 of GenBank No. AQT34120.1, or the amino acid sequence that is at least 90%, 95%, 96%, 97%, 98% or 99% identical thereto.

In some embodiments, the amino acid sequence of the gE is set forth as SEQ ID NO:4: MGTVNKPVVG VLMGFGIITG TLRITNPVRA SVLRYDDFHI DEDKLDTNSV YEPYYHSDHA ESSWVNRGES SRKAYDHNSP YIWPRNDYDG FLENAHEHHG VYNQGRGIDS GERLMQPTQM SAQEDLGDDT GIHVIPTLNG DDRHKIVNVD QRQYGDVFKG DLNPKPQGQR LIEVSVEENH PFTLRAPIQR IYGVRYTETW SFLPSLTCTG DAAPAIQHIC LKHTTCFQDV VVDVDCAENT KEDQLAEISY RFQGKKEADQ PWIVVNTSTL FDELELDPPE IEPGVLKVLR TEKQYLGVYI WNMRGSDGTS TYATFLVTWK GDEKTRNPTP AVTPQPRGAE FHMWNYHSHV FSVGDTFSLA MHLQYKIHEA PFDLLLEWLY VPIDPTCQPM RLYSTCLYHP NAPQCLSHMN SGCTFTSPHL AQRVASTVYQ NCEHADNYTA YCLGISHMEP SFGLILHDGG TTLKFVDTPE SLSGLYVFVV YFNGHVEAVA YTVVSTVDHF VNAIEERGFP PTAGQPPATT KPKEITPVNP GTSPLIRYAA WTGGLA. In some embodiments, the gE antigen comprises the amino acid sequence of SEQ ID NO:4, or the amino acid sequence that is at least 90%, 95%, 96%, 97%, 98% or 99% identical to SEQ ID NO:4.

In some embodiments, the amino acid sequence of the gE is set forth as SEQ ID NO:5: MGTVNKPVVG VLMGFGIITG TLRITNPVRA SVLRYDDFHT DEDKLDTNSV YEPYYHSDHA ESSWVNRGES SRKAYDHNSP YIWPRNDYDG FLENAHEHHG VYNQGRGIDS GERLMQPTQM SAQEDLGDDT GIHVIPTLNG DDRHKIVNVD QRQYGDVFKG DLNPKPQGQR LIEVSVEENH PFTLRAPIQR IYGVRYTETW SFLPSLTCTG DAAPAIQHIC LKHTTCFQDV VVDVDCAENT KEDQLAEISY RFQGKKEADQ PWIVVNTSTL FDELELDPPE IEPGVLKVLR TEKQYLGVYI WNMRGSDGTS TYATFLVTWK GDEKTRNPTP AVTPQPRGAE FHMWNYHSHV FSVGDTFSLA MHLQYKIHEA PFDLLLEWLY VPIDPTCQPM RLYSTCLYHP NAPQCLSHMN SGCTFTSPHL AQRVASTVYQ NCEHADNYTA YCLGISHMEP SFGLILHDGG TTLKFVDTPE SLSGLYVFVV YFNGHVEAVA YTVVSTVDHF VNAIEERGFP PTAGQPPATT KPKEITPVNP GTSPLLR. In some embodiments, the gE antigen comprises the amino acid sequence of SEQ ID NO:5, or the amino acid sequence that is at least 90%, 95%, 96%, 97%, 98% or 99% identical to SEQ ID NO:5.

A unit dose of the immunogenic composition, which is typically a 0.5 ml dose, may comprise from about 10 μg to about 100 μg of the gE antigen, preferably from about 25 μg to about 75 μg of the gE antigen, preferably about 40 to about 60 μg of the gE antigen, or about 50 μg of the gE antigen.

### C. Additional Components

The immunogenic compositions of the present disclosure may comprise one or more additional components, such as one or more excipients, another adjuvant, and/or additional antigens.

### 1. Excipients

Pharmaceutically acceptable excipients of the present disclosure include for instance, solvents, bulking agents, buffering agents, tonicity adjusting agents, and preservatives (Pramanick et al., Pharma Times, 45:65-77, 2013). In some embodiments the immunogenic compositions may comprise an excipient that functions as one or more of a solvent, a bulking agent, a buffering agent, and a tonicity adjusting agent (e.g., sodium chloride in saline may serve as both an aqueous vehicle and a tonicity adjusting agent).

In some embodiments, the immunogenic compositions comprise an aqueous vehicle as a solvent. Suitable vehicles include for instance sterile water, saline solution, phosphate buffered saline, and Ringer's solution. In some embodiments, the composition is isotonic.

The immunogenic compositions may comprise a buffering agent. Buffering agents control pH to inhibit degradation of the active agent during processing, storage and optionally reconstitution. Suitable buffers include for instance salts comprising acetate, citrate, phosphate or sulfate. Other suitable buffers include for instance amino acids such as arginine, glycine, histidine, and lysine. The buffering agent may further comprise hydrochloric acid or sodium hydroxide. In some embodiments, the buffering agent maintains the pH of the composition within a range of 6 to 9. In some embodiments, the pH is greater than (lower limit) 6, 7 or 8. In some embodiments, the pH is less than (upper limit) 9, 8, or 7. That is, the pH is in the range of from about 6 to 9 in which the lower limit is less than the upper limit.

The immunogenic compositions may comprise a tonicity adjusting agent. Suitable tonicity adjusting agents include for instance dextrose, glycerol, sodium chloride, glycerin and mannitol.

The immunogenic compositions may comprise a bulking agent. Bulking agents are particularly useful when the pharmaceutical composition is to be lyophilized before administration. In some embodiments, the bulking agent is a protectant that aids in the stabilization and prevention of degradation of the active agents during freeze or spray drying and/or during storage. Suitable bulking agents are sugars (mono-, di- and polysaccharides) such as sucrose, lactose, trehalose, mannitol, sorbital, glucose and raffinose.

The immunogenic compositions may comprise a preservative. Suitable preservatives include for instance antioxidants and antimicrobial agents. However, in preferred embodiments, the immunogenic composition is prepared under sterile conditions and is in a single use container, and thus does not necessitate inclusion of a preservative.

### 2. Additional Adjuvants

Adjuvants are known in the art and include, but are not limited to, alum (aluminum salts), oil-in-water emulsions, water-in-oil emulsions, liposomes, and microparticles, such as poly(lactide-co-glycolide) microparticles (Shah et al., Methods Mol Biol, 1494:1-14, 2017). In some embodiments, the immunogenic compositions further comprises an aluminum salt adjuvant to which the gE antigen is adsorbed. In some embodiments, the aluminum salt adjuvant comprises one or more of the group consisting of amorphous aluminum hydroxyphosphate sulfate, aluminum hydroxide, aluminum phosphate, and potassium aluminum sulfate. In some embodiments, the aluminum salt adjuvant comprises one or both of aluminum hydroxide and aluminum phosphate. In some embodiments, the aluminum salt adjuvant consists of aluminum hydroxide. In some embodiments, a unit dose (e.g., about 0.5 ml) of the immunogenic composition comprises from about 0.25 to about 0.50 mg Al³⁺, preferably from about 0.30 to about 0.40 mg Al³⁺.

In other embodiments, the immunogenic composition further comprises an additional adjuvant. Additional suitable adjuvants include, but are not limited to, squalene-in-water emulsions (e.g., MF59 or AS03), TLR3 agonists (e.g., poly-IC or poly-ICLC), TLR4 agonists (e.g., saponins such as Quil A or QS-21, as in ASO1 or AS02), TLR5 agonists (bacterial flagellin), and TLR7 and/or TLR8 agonists (imidazoquinoline derivatives such as imiquimod, and resiquimod)(Coffman et al., Immunity, 33:492-503, 2010). For veterinary use and for production of antibodies in non-human animals, mitogenic components of Freund's adjuvant (both complete and incomplete) can be used.

### D. Kits

The present disclosure also provides kits comprising: i) an immunogenic composition comprising a VZV gE antigen and a toll-like receptor 9 (TLR9) agonist, such as a CpG oligonucleotide; and ii) a set of instructions for administration of the immunogenic composition to stimulate an immune response against the gE antigen in a mammalian subject, such as a human subject in need thereof. Additionally, the present disclosure provides kits comprising: i) a first composition comprising a VZV gE antigen; ii) a second composition comprising a TLR9 agonist, such as a CpG oligonucleotide; iii) instructions for mixing the first composition with the second composition to prepare an immunogenic composition; and optionally iv) a further set of instructions for administration of the immunogenic composition to stimulate an immune response against the gE antigen in a mammalian, such as a human subject in need thereof. In some embodiments, the CpG oligonucleotide comprises the sequence 5′-AACGTTCG-3′. In some embodiments, the CpG oligonucleotide comprises the sequence 5′-AACGTTCGAG-3′ (SEQ ID NO:3). In some preferred embodiments, the CpG oligonucleotide comprises the sequence of 5′-TGACTGTGAA CGTTCGAGAT GA-3′ (SEQ ID NO:1).

The kits may comprise an immunogenic composition packaged appropriately. For example, if the immunogenic composition is a freeze-dried power, a vial with a resilient stopper is normally used so that the powder may be easily resuspended by injecting fluid (e.g., sterile water, saline, etc.) through the resilient stopper. In some embodiments, the kits comprise a device for administration (e.g., syringe and needle). The instructions relating to the use of the immunogenic composition generally include information as to dosage, schedule and route of administration for the intended methods of use. In some embodiments, the immunogenic compositions are for stimulating an immune response against VZV.

### II. Methods Of Use

The present disclosure relates to methods for stimulating an immune response against VZV, comprising: administering an immunogenic composition comprising a VZV glycoprotein E (gE) antigen and a Toll-like receptor 9 (TLR9) agonist, such as a CpG oligonucleotide, to a mammalian subject so as to stimulate an immune response against the gE antigen in the mammalian subject. In preferred embodiments, the immunogenic compositions are to be administered by intramuscular injection, optionally in a volume of about 0.5 mL (e.g., unit dose). In some embodiments, the intramuscular injection is into the deltoid muscle of the upper arm of a human subject in need thereof.

Stimulating an immune response, means increasing the immune response, which can arise from eliciting a de novo immune response (e.g., as a consequence of an initial vaccination regimen) or enhancing an existing immune response (e.g., as a consequence of a booster vaccination regimen). In some embodiments, stimulating an immune response includes but is not limited to one or more of the group consisting of: stimulating cytokine production; stimulating B lymphocyte proliferation; stimulating antibody production; stimulating interferon pathway-associated gene expression; stimulating chemoattractant-associated gene expression; and stimulating plasmacytoid dendritic cell maturation. In some preferred embodiments, stimulating an immune response comprises increasing an antigen-specific antibody response in the subject.

### ENUMERATED EMBODIMENTS

1. An immunogenic composition for stimulating an immune response against a varicella zoster virus (VZV), comprising a VZV glycoprotein E (gE) antigen and a toll-like receptor 9 (TLR9) agonist, wherein the TLR9 agonist is an oligonucleotide of from 10 to 35 nucleotides in length comprising an unmethylated cytidine-phospho-guanosine (CpG) motif, and the gE antigen and the oligonucleotide are present in the immunogenic composition in amounts effective to stimulate an immune response against the gE antigen in a mammalian subject.
2. The composition of embodiment 1, wherein the oligonucleotide comprises the sequence 5′-AACGTTCGAG-3′ (SEQ ID NO:3).
3. The composition of embodiment 1, wherein the oligonucleotide comprises the sequence of 5′-TGACTGTGAA CGTTCGAGAT GA-3′(SEQ ID NO:1).
4. The composition of any one of embodiments 1-3, wherein the oligonucleotide comprises a modified nucleoside, optionally wherein the modified nucleoside is selected from the group consisting of 2′-deoxy-7-deazaguanosine, 2′-deoxy-6-thioguanosine, arabinoguanosine, 2′-deoxy-2′substituted-arabinoguanosine, and 2′-O-substituted-arabinoguanosine.
5. The composition of embodiment 4, wherein the oligonucleotide comprises the sequence 5′-TCG₁AACG₁TTCG₁-3′ (SEQ ID NO:2) in which G₁ is 2′-deoxy-7-deazaguanosine, optionally wherein the oligonucleotide comprises the sequence 5′-TCG₁AACG₁TTCG₁-X-G₁CTTG₁CAAG₁CT-5′, and in which G₁ is 2′-deoxy-7-deazaguanosine and X is glycerol (5′-SEQ ID NO:2-3′-X-3′-SEQ ID NO:2-5′).
6. The composition of any one of embodiments 1-5, wherein the oligonucleotide comprises at least one phosphorothioate linkage, optionally wherein all nucleotide linkages are phosphorothioate linkages.
7. The composition of any one of embodiments 1-6, wherein the oligonucleotide is a single-stranded oligodeoxynucleotide.
8. The composition of any one of embodiments 1-7, wherein a 0.5 ml dose of the immunogenic composition comprises from about 375 μg to about 6000 μg of the oligonucleotide or from about 750 μg to about 3000 μg of the oligonucleotide, optionally wherein a 0.5 ml dose of the immunogenic composition comprises about 375 μg, about 750 μg, about 1500 μg, about 3000 μg, or 6000 μg about of the oligonucleotide.
9. The composition of any one of embodiments 1-8, wherein the VZV gE antigen is a truncated, recombinant protein devoid of signal peptide, transmembrane and cytoplasmic domains of a full-length VZV gE antigen, optionally wherein the VZV gE antigen comprises:
(i) the amino acid sequence of residues 39-585 of GenBank No. AQT34120.1, or the amino acid sequence that is at least 90%, 95%, 96%, 97%, 98% or 99% identical thereto; or (ii) the amino acid sequence of SEQ ID NO:4, or the amino acid sequence that is at least 90%, 95%, 96%, 97%, 98% or 99% identical to SEQ ID NO:4; or (iii) the amino acid sequence of SEQ ID NO:5, or the amino acid sequence that is at least 90%, 95%, 96%, 97%, 98% or 99% identical to SEQ ID NO:5.
10. The composition of any one of embodiments 1-9, wherein a 0.5 ml dose of the immunogenic composition comprises from about 25 to about 75 μg of the gE antigen, optionally wherein the immunogenic composition comprises from about 40 to about 60 μg of the gE antigen, or about 50 μg of the gE antigen.
11. The composition of any one of embodiments 1-10, further comprising an aluminum salt adjuvant.
12. The composition of embodiment 11, wherein the aluminum salt adjuvant comprises one or more of the group consisting of amorphous aluminum hydroxyphosphate sulfate, aluminum hydroxide, aluminum phosphate, and potassium aluminum sulfate.
13. The composition of embodiment 11, wherein the aluminum salt adjuvant comprises aluminum hydroxide.
14. The composition of any one of embodiments 11-13, wherein a 0.5 ml dose of the immunogenic composition comprises from about 0.25 to about 0.50 mg Al³⁺, optionally wherein a 0.5 ml dose of the immunogenic composition comprises from about 0.30 to about 0.40 mg Al³⁺.
15. The composition of any one of embodiments 1-14, wherein the mammalian subject is a human subject.
16. A kit comprising:
i) the immunogenic composition of any one of embodiments 1-15, and
ii) instructions for administration of the composition to stimulate an immune response against the gE antigen in the mammalian subject.
17. A kit comprising:
i) a first composition comprising glycoprotein E (gE) antigen of a varicella zoster virus;
ii) a second composition comprising a TLR9 agonist; and
iii) instructions for mixing the first composition with the second composition to prepare an immunogenic composition.
18. The kit of embodiment 17, further comprising:
iv) a further set of instructions for administration of the immunogenic composition to stimulate an immune response against the gE antigen in a mammalian subject.
19. The kit of any one of embodiments 16-18, further comprising a syringe and needle for intramuscular injection of the immunogenic composition.
20. A method for stimulating an immune response against varicella zoster virus (VZV) in a mammalian subject, comprising administering the immunogenic composition of any one of embodiments 1-15 to a mammalian subject so as to stimulate an immune response against the gE antigen in the mammalian subject.
21. The method of embodiment 20, wherein the immunogenic composition is administered by intramuscular injection.

### EXAMPLES

Abbreviations: CpG (unmethylated cytidine-phospho-guanosine); CTRL (control); gE (VZV glycoprotein E); LAV (live attenuated virus); mcg (microgram); mcl (microliter); MPL (monophosphoryl lipid A); TLR9 (toll-like receptor 9); and VZV (varicella zoster virus).

### Example 1

### Immunogenicity of CpG-Adjuvanted Zoster Vaccine in Mice

This example provides a description of a preclinical study to assess immunogenicity of zoster vaccines in mice infected with a live, attenuated varicella zoster virus (VZV) strain (referred to below as LAV).

Antigen: Recombinant VZV glycoprotein E (gE) is obtained by cell culture of genetically engineered Chinese Hamster Ovary host cells expressing a truncated version of gE lacking the transmembrane anchor and carboxy-terminal domain, and is therefore secreted into the supernatant. The cell culture is supported by media containing amino acids, but no albumin, antibiotics or animal derived proteins. gE is purified by chromatography and lyophilized for future use as described (Haumont et al., Virus Res, 40:199-204, 1996). For initial pre-clinical studies, VZV gE was obtained commercially.

Vaccines were prepared before use by combining gE with either an adjuvant formulation or a pharmaceutically acceptable buffer or normal saline. Specifically, gE was adsorbed to alum (aluminum hydroxide) for 30 min prior to injection, and ASO1B and CpG 1018 adjuvants were each mixed with gE immediately prior to injections.

Experimental Design: The adjuvant activity of CpG 1018 was assessed in C57BL/6 mice (n=4-8/group) primed with a live attenuated VZV vaccine (104plaque forming units of LAV in 500 mcl administered by the subcutaneous route) 35 days prior (day −35) to the start of the immunization schedule. The LAV used was the VARIVAX® varicella virus vaccine live marketed by Merck & Co., Inc. (Whitehouse Station, NJ). As mice do not support sufficient replication of VZV, this was done to mimic existing VZV infection in humans. Mice were immunized with 50 mcl of a vaccine formulation or a saline control by the intramuscular route on days 0 and 28. Groups, dosing amounts and sampling are listed in Table 1-1.

TABLE 1-1
Vaccination Groups for Evaluation of gE Immunogenicity

| | LAV Vaccine | Subunit Vaccine | # | | |
|---|---|---|---|---|---|
| Group | (Day-35) | (Day 0 and 28) | Mice | Bleed(s) | Sacrifice |
| 1 | None | None | 4 | Day 56 | Day 56 |
| | | (Saline only control) | | | |
| 2 | LAV | None | 8 | Day 21, | Day 56 |
| | | (LAV only control) | | 56 | |
| 3 | LAV | gE (5 mcg) | 8 | Day 21, | Day 56 |
| | | | | 56 | |
| 4 | LAV | gE (5 mcg) + | 8 | Day 21, | Day 56 |
| | | alum (50 mcg) | | 56 | |
| 5 | LAV | gE (5 mcg) + | 8 | Day 21, | Day 56 |
| | | AS01b (50 mcL) | | 56 | |
| 6 | LAV | gE (5 mcg) + | 8 | Day 21, | Day 56 |
| | | CpG 1018 (10 mcg) | | 56 | |
| 7 | LAV | gE (5 mcg) + | 8 | Day 21, | Day 56 |
| | | CpG 1018 (10 mcg) + | | 56 | |
| | | alum (50 mcg) | | | |

VZV gE contains both B cell and T cell epitopes. Accordingly, serum antibody responses to gE were measured with a commercially available ELISA using VZV gE antigen-coated microtiter plates following administration of 1 (day 21) or 2 doses (day 56) of vaccine. CD3⁺CD4⁺ T cell cytokine responses (IL-2 and IFN-γ) were measured by flow cytometry after in vitro re-stimulation of spleen cell cultures with overlapping peptides spanning the length of the gE antigen. Three spleen cell sub-pools per group were tested (3, 3, or 2 mouse spleens/pool). T cell responses were evaluated as percentage of CD4⁺ T cells or as a percentage of CD154⁺CD4⁺ T cells, where CD154 is a molecule transiently up-regulated in antigen-stimulated cells and thus serves as a specific marker of antigen-reactive CD4⁺ T cells (i.e., cells responding to gE peptide stimulation ex vivo). Mice were monitored for health parameters, including injection site reactions, and general appearance.

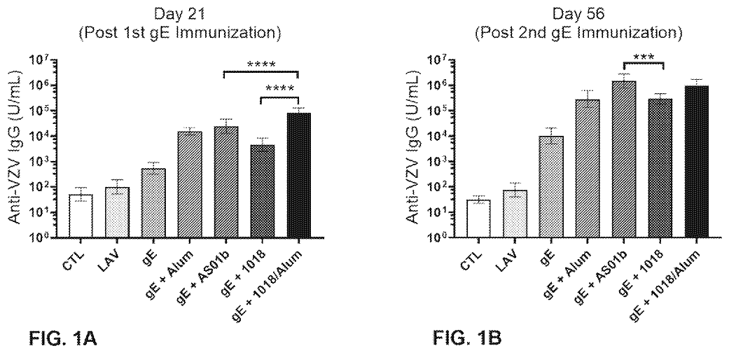

All adjuvant groups tested induced high levels of anti-VZV IgG response after prime and booster vaccinations. On Day 21 post 1^{st} gE immunization (prime), anti-VZV IgG responses were elevated compared to background levels observed in non-immunized mice and mice that were LAV-primed but not further immunized. Addition of the adjuvants aluminum hydroxide (alum) alone or CpG 1018 alone increased the anti-VZV IgG response compared to immunization with gE antigen alone. The highest anti-VZV IgG responses post 1^{st} immunization was observed in mice immunized with CpG-1018+alum (FIG. 1A). Responses in the gE+CpG 1018+alum immunized group were significantly higher than in mice immunized with gE in combination with CpG-1018 alone or mice immunized with gE+AS01b. Anti-VZV IgG responses were observed to be about one log higher post 2^{nd} immunization (boost) on Day 56 in all gE±adjuvants groups (FIG. 1i), at which point comparable responses were seen in gE+AS01b and gE+CpG 1018+alum groups and the response in gE+AS01b immunized mice was significantly higher than in mice immunized with gE+CpG 1018 (ANOVA with Tukey's multiple comparisons test, ***P<0.001, ****P<0.0001).

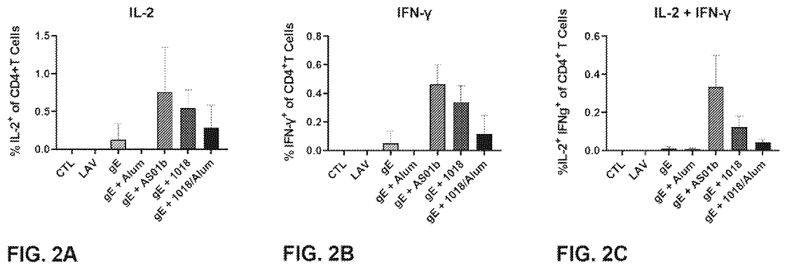

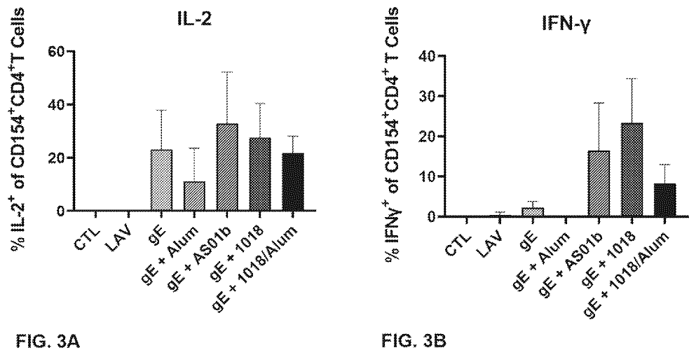

Ex vivo intracellular cytokine expression responses in gE peptides-stimulated spleen cells were quantified as a measure of the effect of immunization regimens on gE antigen-specific CD4⁺ T cell responses. gE-stimulated cytokine expression was measured as a percentage of all CD4⁺ T cells and as a percentage of the antigen-reactive population (CD154⁺CD4⁺ T cells). IL-2 and IFN-γ expression was absent or at very low levels in CD4⁺ T cells from the control (CTL), LAV-primed only, gE only and alum adjuvanted groups. In contrast, use of AS01b or CpG 1018 as adjuvants induced comparably elevated IL-2 (FIG. 2A) and IFN-γ expressing (FIG. 2B) T cells as measured after ex vivo re-stimulation. IL-2 and IFN-γ expression trended lower in re-stimulated CD4⁺ T cells from gE+CpG 1018+alum-immunized groups. IL-2 and IFN-γ co-expressing CD4⁺ T cells were highest in cells from AS01b-adjuvanted mice, followed by cells from CpG 1018 and CpG 1018+alum-adjuvanted mice (FIG. 2C). The pattern of response was similar within the antigen-reactive (CD154⁺) CD4⁺ T cell populations with comparable cytokine expression in gE peptides re-stimulated T cells from AS01b and CpG 1018-adjuvanted mice (FIG. 3A-3B). IFN-γ expression trended higher in antigen-specific T cells from CpG 1018 adjuvanted mice, as compared to T cells from AS01b adjuvanted mice, and both intracellular IL-2 and IFN-γ expression were lower in mice in which CpG 1018+alum was used to adjuvant gE responses.

In summary, all adjuvants tested induced high levels of anti-VZV IgG responses in C57BL/6 mice with the highest antibody responses in gE+CpG 1018+alum immunized mice after a first immunization, and in gE+ASOlb-immunized mice after the booster immunization, although the levels induced in CpG 1018+alum were comparable. CD4⁺ T cell responses to gE immunizations were evaluated following ex vivo gE peptides re-stimulations and the highest responses were observed in the CpG 1018 and ASOlb-adjuvanted groups when measured as a percentage of all CD4⁺ cells of the antigen-reactive population, as identified by expression of CD154. The data demonstrated that CpG 1018 in combination with alum or CpG 1018 as a stand-alone adjuvant induced comparable antibody and T cell responses, respectively, to AS01b in immunized mice. As T cell responses are believed to be primarily responsible for control of shingles outbreaks, the data support use of CpG 1018 as an adjuvant in a gE-based vaccine. CpG 1018 alone or in combination with alum is contemplated to generate a favorable reactogenicity profile in comparison to AS01b.

Although the foregoing disclosure has been described in some detail by way of illustration and example for purposes of clarity and understanding, it will be apparent to those skilled in the art that certain changes and modifications may be practiced. Therefore, the examples should not be construed as limiting the scope of the disclosure, which is delineated by the appended claims.

We claim:

1. An immunogenic composition for stimulating an immune response against a varicella zoster virus (VZV), comprising a VZV glycoprotein E (gE) antigen and a toll-like receptor 9 (TLR9) agonist, wherein the TLR9 agonist is an oligonucleotide of from 10 to 35 nucleotides in length comprising an unmethylated cytidine-phospho-guanosine (CpG) motif, and the gE antigen and the oligonucleotide are present in the immunogenic composition in amounts effective to stimulate an immune response against the gE antigen in a mammalian subject.

2. The immunogenic composition of claim 1, wherein the oligonucleotide comprises the sequence 5′-AACGTTCGAG-3′ (SEQ ID NO:3).

3. The immunogenic composition of claim 1, wherein the oligonucleotide comprises the sequence of 5′-TGACTGTGAA CGTTCGAGAT GA-3′(SEQ ID NO:1).

4. The immunogenic composition of claim 1, wherein the oligonucleotide comprises a modified nucleoside, optionally wherein the modified nucleoside is selected from the group consisting of 2′-deoxy-7-deazaguanosine, 2′-deoxy-6-thioguanosine, arabinoguanosine, 2′-deoxy-2′substituted-arabinoguanosine, and 2′-O-substituted-arabinoguanosine.

5. The immunogenic composition of claim 4, wherein the oligonucleotide comprises the sequence 5′-TCG₁AACG₁TTCG₁-3′ (SEQ ID NO:2) in which G₁ is 2′-deoxy-7-deazaguanosine, optionally wherein the oligonucleotide comprises the sequence
5′-TCG₁AACG₁TTCG₁-X-G₁CTTG₁CAAG₁CT-5′, and in which G₁ is 2′-deoxy-7-deazaguanosine and X is glycerol (5′-SEQ ID NO:2-3′-X-3′-SEQ ID NO:2-5′).

6. The immunogenic composition of claim 3, wherein the oligonucleotide comprises at least one phosphorothioate linkage, or wherein all nucleotide linkages are phosphorothioate linkages.

7. The immunogenic composition of claim 6, wherein the oligonucleotide is a single-stranded oligodeoxynucleotide.

8. The immunogenic composition of claim 6, wherein a 0.5 ml dose of the immunogenic composition comprises from about 375 μg to about 6000 μg of the oligonucleotide or from about 750 μg to about 3000 μg of the oligonucleotide, or wherein a 0.5 ml dose of the immunogenic composition comprises about 375 μg, about 750 μg, about 1500 μg, about 3000 μg, or 6000 μg about of the oligonucleotide.

9. The immunogenic composition of claim 8, wherein the VZV gE antigen is a truncated, recombinant protein devoid of signal peptide, transmembrane and cytoplasmic domains of a full-length VZV gE antigen.

10. The immunogenic composition of claim 9, wherein a 0.5 ml dose of the immunogenic composition comprises from about 25 to about 75 μg of the gE antigen, or wherein the immunogenic composition comprises from about 40 to about 60 μg of the gE antigen, or about 50 μg of the gE antigen.

11. The immunogenic composition of claim 10, further comprising an aluminum salt adjuvant.

12. The immunogenic composition of claim 11, wherein the aluminum salt adjuvant comprises one or more of the group consisting of amorphous aluminum hydroxyphosphate sulfate, aluminum hydroxide, aluminum phosphate, and potassium aluminum sulfate.

13. The immunogenic composition of claim 11, wherein the aluminum salt adjuvant comprises aluminum hydroxide.

14. The immunogenic composition of claim 11, wherein a 0.5 ml dose of the immunogenic composition comprises from about 0.25 to about 0.50 mg Al³⁺, or wherein a 0.5 ml dose of the immunogenic composition comprises from about 0.30 to about 0.40 mg Al³⁺.

15. The immunogenic composition of any one of claims 1-14, wherein the mammalian subject is a human subject.

16. A kit comprising:
i) the immunogenic composition of claim 15, and
ii) instructions for administration of the immunogenic composition to stimulate an immune response against the gE antigen in the mammalian subject.

17. A kit comprising:
i) a first composition comprising glycoprotein E (gE) antigen of a varicella zoster virus;
ii) a second composition comprising a TLR9 agonist; and
iii) instructions for mixing the first composition with the second composition to prepare an immunogenic composition.

18. The kit of claim 17, further comprising:
iv) a further set of instructions for administration of the immunogenic composition to stimulate an immune response against the gE antigen in a mammalian subject.

19. The kit of claim 18, further comprising a syringe and needle for intramuscular injection of the immunogenic composition.

20. A method for stimulating an immune response against varicella zoster virus (VZV) in a mammalian subject, comprising administering the immunogenic composition of any one of claims 1-15 to a mammalian subject so as to stimulate an immune response against the gE antigen in the mammalian subject.

21. The method of claim 20, wherein the immunogenic composition is administered by intramuscular injection.
